SEQUENCE LISTING (2 sequences)

SEQ ID NO: 1 - DNA, 978 bp, Artificial Sequence
  Human BMI-1 cDNA fragment
atgcatcgaacaacgagaatcaagatcactgagctaaatccccacctgatgtgtgtgctttgtggagggtacttcattgatgccacaaccataatagaatgtctacattccttctgtaaaacgtgtattgttcgttacctggagaccagcaagtattgtcctatttgtgatgtccaagttcacaagaccagaccactactgaatataaggtcagataaaactctccaagatattgtatacaaattagttccagggcttttcaaaaatgaaatgaagagaagaagggatttttatgcagctcatccttctgctgatgctgccaatggctctaatgaagatagaggagaggttgcagatgaagataagagaattataactgatgatgagataataagcttatccattgaattctttgaccagaacagattggatcggaaagtaaacaaagacaaagagaaatctaaggaggaggtgaatgataaaagatacttacgatgcccagcagcaatgactgtgatgcacttaagaaagtttctcagaagtaaaatggacatacctaatactttccagattgatgtcatgtatgaggaggaacctttaaaggattattatacactaatggatattgcctacatttatacctggagaaggaatggtccacttccattgaaatacagagttcgacctacttgtaaaagaatgaagatcagtcaccagagagatggactgacaaatgctggagaactggaaagtgactctgggagtgacaaggccaacagcccagcaggaggtattccctccacctcttcttgtttgcctagccccagtactccagtgcagtctcctcatccacagtttcctcacatttccagtactatgaatggaaccagcaacagccccagcggtaaccaccaatcttcttttgccaatagacctcgaaaatcatcagtaaatgggtcatcagcaacttcttctggt

SEQ ID NO: 2 - DNA, 6907 bp, Artificial Sequence
  pPB-BMI1, an expression vector with human BMI-1 cDNA insert
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggggcgcgccattctagattaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcaactagaatgctagcatgggcccatctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattcagatctgcttggtaccgagctcggatccactagtccagtgtggtggaattctgcagatatcaacaagtttgtacaaaaaagcaggctccgcggccgcccccttcaccatgcatcgaacaacgagaatcaagatcactgagctaaatccccacctgatgtgtgtgctttgtggagggtacttcattgatgccacaaccataatagaatgtctacattccttctgtaaaacgtgtattgttcgttacctggagaccagcaagtattgtcctatttgtgatgtccaagttcacaagaccagaccactactgaatataaggtcagataaaactctccaagatattgtatacaaattagttccagggcttttcaaaaatgaaatgaagagaagaagggatttttatgcagctcatccttctgctgatgctgccaatggctctaatgaagatagaggagaggttgcagatgaagataagagaattataactgatgatgagataataagcttatccattgaattctttgaccagaacagattggatcggaaagtaaacaaagacaaagagaaatctaaggaggaggtgaatgataaaagatacttacgatgcccagcagcaatgactgtgatgcacttaagaaagtttctcagaagtaaaatggacatacctaatactttccagattgatgtcatgtatgaggaggaacctttaaaggattattatacactaatggatattgcctacatttatacctggagaaggaatggtccacttccattgaaatacagagttcgacctacttgtaaaagaatgaagatcagtcaccagagagatggactgacaaatgctggagaactggaaagtgactctgggagtgacaaggccaacagcccagcaggaggtattccctccacctcttcttgtttgcctagccccagtactccagtgcagtctcctcatccacagtttcctcacatttccagtactatgaatggaaccagcaacagccccagcggtaaccaccaatcttcttttgccaatagacctcgaaaatcatcagtaaatgggtcatcagcaacttcttctggttatccatatgacgtcccagactatgcctgaaagggtgggcgcgccgacccagctttcttgtacaaagtggttgatatccagcacagtggcggccgctcgagatgcatcgatgatctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccattcatgaatgaattcatgtcgacatactagttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaatctagtatacgcgtatgcggccgcttaattaatccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

(19) United States
(12) Patent Application Publication
(10) Pub. No.: US 2020/0268839 A1
(43) Pub. Date: Aug. 27, 2020

(54) COMPOSITION FOR TREATING PULMONARY FIBROSIS AND EMPHYSEMA AND THERAPEUTIC METHOD USING THE SAME

(71) Applicant: National Yang-Ming University

(72) Inventors: Cheng-Wen WU, Taipei City (TW); Erh-Hsuan LIN, Taipei City (TW); Ching-Huei LIN, Taipei City (TW)

(21) Appl. No.: 16/798,953

(22) Filed: Feb. 24, 2020

Related U.S. Application Data
(63) Continuation of application No. 62/809,949, filed on Feb. 25, 2019.

(51) Int. Cl. A61K 38/17; A61K 47/34; A61P 11/00

(52) U.S. Cl. CPC A61K 38/1709; A61P 11/00; A61K 47/34

(57) ABSTRACT

The present disclosure provides a nucleic acid fragment, a pharmaceutical composition, and a therapeutic process for treating a subject having chronic obstructive pulmonary disease (COPD). Especially, the nucleic acid fragment, the pharmaceutical composition, and the therapeutic process are therapeutic-efficient for treating pulmonary fibrosis and emphysema of the subject, as demonstrated in this disclosure.

### BACKGROUND OF THE INVENTION

### Field of Invention

The present invention relates to a nucleic acid fragment, a pharmaceutical composition, and a therapeutic process for treating a subject having chronic obstructive pulmonary disease (COPD).

### Related Art

Chronic obstructive pulmonary disease (COPD) is a progressive and chronic disease that causes breathing problems. It is a major public health problem with a high and growing prevalence and currently rated the fourth most common specific cause of death globally, and it is predicted to be the third by 2030. Long-term exposure to irritating substances, such as cigarette smoke and atmospheric particulate matters (e.g., PM₁₀ and/or PM_{2.5}), may cause a chronic inflammatory response in the lungs, leading to irreversible injuries to lung tissue (mostly due to fibrosis caused by inappropriate repair) and resulting in impairment of gas exchange and poor airflow from the lungs. COPD is characterized by a mixture of small airways disease (chronic bronchitis) and parenchymal destruction (emphysema). The pharmaceutical approach to restore the disrupted pulmonary functions caused by COPD is currently unavailable. Until now, COPD can be only managed to relieve the symptoms and delay the progression of the disease. Patients suffering from late stage of COPD may need surgical treatments, even lung transplantation.

Recently, regenerative approaches including stem cell therapy have shed some lights to the COPD therapy. Stem cell based therapies aim to replace the damaged cells with immature cells (either autologous or exogenous) which potentially can differentiate into several types of lung cells, such as the alveolar epithelial cells, so as to restore the functions destroyed by the inappropriate repair of lung tissue after injury. Several researches of stem cell therapies have been demonstrated successful effects in COPD animal models. However, many human trials have failed in showing any benefits. It is speculated the failure is owing to the complexity of cell source and lung microenvironment, whether transplanted cells have differentiated for reconstitution of airway/alveolar epithelium are questioned. It is also uncertain whether the transplanted cells are able to repair, to slow down, or to prevent the disease. Furthermore, safety issues have been raised recently concerning the use of stem cells in vivo.

BMI-1 (B lymphoma Mo-MLU insertion region 1 homolog) gene encodes a ring finger protein, polycomb complex protein BMI-1, also known as polycomb group RING finger protein 4 (PCGF4) or RING finger protein 51 (RNF51). The polycomb complex protein BMI-1 and RING 1A/B protein constitute the catalytic RING domain subunit of the polycomb repressive complex 1 (PRC1), which is an epigenetic repressor in response to DNA damage and functions through chromatin remodeling. BMI-1 is found to be an oncogene. Knockout of BMI-1 gene has been proved resulting in defects in hematopoiesis and abnormal development of skeletons and brains. Overexpression of BMI-1 is involved with the development of several types of cancer, including prostate cancers, breast cancers, colorectal cancers and lymphomas. Inhibiting BMI-1 seems to reduce the resistance of cancer to chemotherapy and has been shown to reduce the self-renewal of the colorectal cancer stem cells. However, the relationship between BMI-1 and COPD is still unclear.

### SUMMARY OF THE INVENTION

Therefore, the present invention aims to provide a pharmaceutical composition and a therapeutic process for treating a subject with chronic obstructive pulmonary disease (COPD).

In view of the foregoing objectives, the invention provides a nucleic acid fragment for treating a subject having chronic obstructive pulmonary disease (COPD), comprising a cDNA fragment which encodes BMI-1.

To achieve the above objective, the present disclosure also provides a pharmaceutical composition for treating a subject having chronic obstructive pulmonary disease (COPD). The pharmaceutical composition comprises a cDNA fragment which encodes BMI-1 and a pharmaceutically acceptable carrier which is pulmonary-targeted.

To achieve the above objective, the present disclosure also provides a method for treating a subject having chronic obstructive pulmonary disease (COPD). The method comprises the step(s) of: delivering a therapeutically-effective amount of a pharmaceutical composition to the subject. The pharmaceutical composition comprises a cDNA fragment which encodes BMI-1 and a pharmaceutically acceptable carrier which is pulmonary-targeted.

In one embodiment, the cDNA fragment encodes human BMI-1.

In one embodiment, the cDNA fragment has a sequence of SEQ ID NO: 1.

In one embodiment, the cDNA fragment has a sequence of SEQ ID NO: 2.

In one embodiment, the subject is a mammal.

In one embodiment, the subject is selected from human, primate, hamster, rabbit, rodent, bovine, swine, sheep, horse, goat, canine or feline.

In one embodiment, the nucleic acid fragment, or the pharmaceutical composition, relieves or ameliorates emphysema of the subject.

In one embodiment, the nucleic acid fragment, or the pharmaceutical composition, relieves or ameliorates pulmonary fibrosis of the subject.

In one embodiment, the pharmaceutically-acceptable carrier is a cationic polymer with a molar formula of (C₂H₅N)ₙ, wherein n is an integer of about 10 to 1000.

In one embodiment, the pharmaceutically acceptable carrier is polyethylenimine.

Accordingly, this disclosure uses BMI-1 gene delivery to restore the pulmonary function through inducing proliferation and differentiation of alveolar epithelia cells (AECs) and improving the ratio of slow-cycling cells appeared post-injury. Also, the present invention also discloses that BMI-1 gene delivery significantly reduces the fibrosis induced by Bleomycin in the mouse model.

### BRIEF DESCRIPTION OF THE DRAWINGS

The embodiments will become more fully understood from the detailed description and accompanying drawings, which are given for illustration only, and thus are not limitative of the present invention, and wherein:

FIG. 1A represents a protocol, wherein mice are intratracheally instilled with elastase to induce emphysema syndrome, and then treated with PEI nanoparticles for BMI-1 or control (luc) gene delivery of.

FIGS. 1B and 1C show the pulmonary functions of the mice treated with the protocol as shown in FIG. 1A measured by Plethysmography.

FIG. 1D is Hematoxylin & Eosin staining sections for demonstrating the histopathology of the lungs of the mice treated with the protocol as shown in FIG. 1A.

FIG. 2A exhibits a protocol, wherein mice are intratracheally instilled with elastase and treated with PEI/DNA nanoparticles or PBS. To trace the proliferation of AECs, mice are daily injected with BrdU for a month and then sacrificed for IHC staining of BrdU.

FIG. 2B demonstrates IHC staining of BrdU in lung sections with the treatment as shown in FIG. 2A.

FIG. 2C shows Verhoeff Van Gieson Elastin Staining performed to investigate the

ECM components in lung sections with PEI/BMI-1 treatment and BrdU staining.

FIG. 2D shows immunofluorescent staining of BrdU (red), Pdpn (green, AEC-1 marker), and SPC (white, AEC-2 marker) in lung sections to verify the cell types the proliferated after PEI/BMI-1 treatment.

FIG. 3A demonstrates a protocol, wherein mice are injected with a single dose of BrdU 1 day after injury and PEI nanoparticles treatment to trace the slow-cycling cells, and the mice is sacrificed 6 weeks later.

FIGS. 3B to 3D show immunofluorescent staining of BrdU (red), Pdpn (green, AEC-1 marker), and SPC (white, AEC-2 marker) in lung sections of the mice treated with PBS (FIG. 3B), elastase+PEI/luc (FIG. 3C), and elastase+PEI/BMI-1 (FIG. 3D) by the protocol shown in FIG. 3A.

FIGS. 4A to 4C show the results of the invasive lung function analysis of the mice treated with PEI/DNA nanoparticles.

FIG. 4D is Hematoxylin & Eosin staining sections for demonstrating the histopathology of the lungs of the mice treated with PBS (NT), PEI control (PEI/GFP), and PEI/BMI-1.

FIG. 5A demonstrates a protocol, wherein mice are intratracheally instilled with Bleomycin for inducing pulmonary fibrosis. At day 7, the mice are treated with PEI/DNA nanoparticles. After 14 days, the mice are subjected to lung function analysis and histopathological examination.

FIG. 5B shows the pulmonary functions of the mice treated with the protocol as shown in FIG. 5A measured by whole-body plethysmography. “*” means p value<0.05.

FIG. 6 represents a construction map of the plasmid pPB-hBMI-1 that contains human BMI1 cDNA sequence under the transcriptional regulation of a CAG promoter.

### DETAILED DESCRIPTION OF THE INVENTION

The embodiments of the invention will be apparent from the following detailed description, which proceeds with reference to the accompanying drawings, wherein the same references relate to the same elements.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

For purposes of interpreting this specification, the following definitions will apply and whenever appropriate, terms used in the singular will also include the plural and vice versa. Additional definitions are set forth throughout the detailed description.

The terms “nucleic acid” or “nucleic acid molecule” refer to a deoxyribonucleotide or ribonucleotide polymer in either single- or double-stranded form, and unless otherwise limited, would encompass known analogs of natural nucleotides that can function in a similar manner as naturally occurring nucleotides, such as peptide nucleic acids (PNAs) and locked nucleic acids (LNAs).

The term “cDNA fragment” refers to a fragment of cDNA, normally double stranded, that is derived from an RNA template by reverse transcription. A cDNA fragment is often, but not necessarily, reside in a nucleic acid vector (e.g., a plasmid, phage, etc.). A cDNA fragment can represent a full length mRNA molecule, or some fraction of an mRNA molecule.

The terms “percent (%) sequence identity” or “homology” are defined as the percentage of amino acid residues or nucleotides in a candidate sequence that are identical with the amino acid residues or nucleotides in the reference sequences after aligning the sequences and introducing gaps, if necessary, to achieve the maximum percent sequence identity, and excluding conservative nucleic acid substitutions. Optimal alignment of the sequences for comparison may be produced, besides manually, by means of local homology algorithms known in the art or by means of computer programs which use these algorithms (e.g., BLAST P).

Certain exemplary embodiments according to the present disclosure are described as below.

Nucleic Acid Fragment Encoding BMI-1 for Treating COPD

According to one embodiment of this disclosure, a nucleic acid fragment for treating a subject having chronic obstructive pulmonary disease (COPD) is provided. Said nucleic acid fragment comprises a cDNA fragment encoding BMI-1. The subject can be a mammal, such as a primate, hamster, rabbit, rodent, bovine, swine, sheep, horse, goat, canine or feline, and preferably a human. In the present embodiment, the nucleic acid fragment is isolated from a living organism and preferably an artificial one.

The cDNA fragment preferably encodes the human BMI-1 and has a sequence set forth as SEQ ID NO: 1, representing a full-length BMI-1. Alternatively, the BMI-1 cDNA fragment may have a sequence representing some fraction of BMI-1, but still possess the potency substantially as much as its full-length counterpart. Moreover, the BMI-1 cDNA fragment may have a sequence exhibiting at least 80%, preferable 85%, 90%, 95%, 99%, or 100% (or any percentage in between), sequence identity with SEQ ID NO: 1.

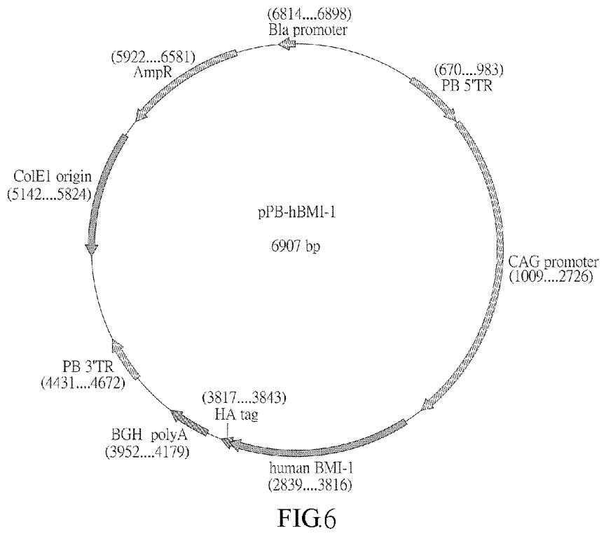

For fulfilling this embodiment, a vector comprising said nucleic acids as described above is also provided. Moreover, a host cell comprising the vector as described above is also provided. In some embodiment, the host cell can be a prokaryotic or eukaryotic cell, preferably artificially selected or genetically engineered. The prokaryotic cell can be an E. coli. The eukaryotic cell can be a COS cell, a 293F cell, a Chinese hamster ovary (CHO) cell or a primarily-cultured alveoli epithelial cell. In addition, the BMI-1 cDNA fragment can have a sequence set forth as SEQ ID NO: 2, an expression vector with BMI-1 cDNA insert as shown in FIG. 6. The plasmid construct, pPB-hBMI-1 as shown in FIG. 6, comprises a CAG promoter and is a vector of mammalian expression type. Similarly, the BMI-1 cDNA fragment, as an expression vector, may have a sequence exhibiting at least 80%, preferable 85%, 90%, 95%, 99%, or 100% (or any percentage in between), sequence identity with SEQ ID NO: 2. The detailed construction of the plasmid with BMI-1 cDNA insert will be discussed in the following experimental examples.

As described above, in this embodiment, the nucleic acid fragment, comprising BMI-1 cDNA, includes polynucleotides substantially identical to SEQ ID NOs: 1 and 2. Moreover, substantially identical sequences may be polymorphic sequences, i.e., alternative sequences or alleles in a population. Substantially identical sequences may also comprise mutagenized sequences, including sequences comprising silent mutations. A mutation may comprise one or more nucleotide residue changes, a deletion of one or more nucleotide residues, or an insertion of one or more additional nucleotide residues. Substantially identical sequences may also comprise various nucleotide sequences encoding for the same amino acid at any given amino acid position in an amino acid sequence of BMI-1, due to the degeneracy of the nucleic acid code.

In general, said nucleic acid fragments, vectors and host cells comprising such nucleic acids can be used to express or generate BMI-1. The nucleic acid fragment of this embodiment can be obtained by any method known in the art. For example, the polynucleotide encoding BMI-1 may be assembled from chemically synthesized oligonucleotides. This would involve, for example and if necessary, the synthesis of overlapping oligonucleotides containing portions of the sequence encoding BMI-1, annealing and ligating those oligonucleotides, and then amplifying the ligated oligonucleotides by PCR.

Pharmaceutical Composition

In another embodiment of this disclosure, a pharmaceutical composition for treating a subject having chronic obstructive pulmonary disease (COPD) is provided. Said pharmaceutical composition comprises a cDNA fragment encoding BMI-1 and a pharmaceutically acceptable carrier which is pulmonary-targeted. Said cDNA fragment, as described above, can be a sequence representing a full-length BMI-1 (e.g., the sequence set forth as SEQ ID NO: 1), or at least some fraction thereof but substantially still possessing its normal function. Alternatively, said cDNA fragment is preferably an expression vector (e.g., the sequence set forth as SEQ ID NO: 2) comprising a BMI-1 cDNA insert and other suitable genetic elements, such as a prompter and/or an enhancer, which may prompt the expression of BMI-1 in a designated host.

Moreover, the pharmaceutically acceptable carrier can be a cationic polymer, such as a polymer with a molar formula of (C₂H₅N)ₙ, and n is an integer of about 10 to 1000. Preferably, the cationic polymer is polyethylenimine (PEI). PEI is a cationic polymer with repeating units composed of an amine group and a two-carbon aliphatic spacer, with the molecular formula (C₂H₅N)ₙ. As evidenced previously, PEI can be in linear or branched forms, and both are efficient for gene delivery in vivo, with alveolar epithelial cells as major targets. When mixing together, PEI and DNA fragments may form nanoparticles and target to lungs. As shown in the following experimental examples, the nucleic acid fragment (e.g., BMI-1 cDNA fragment), and/or the pharmaceutical composition, can relieve and/or ameliorate emphysema and lung fibrosis in the animal model.

Similarly, the composition, variation or connection relationship to other elements of each detail elements of the cDNA fragment encoding BMI-1 can refer to the previous embodiments, and they are not repeated here.

Therapeutic Method for Treating COPD

In another embodiment of this disclosure, a method for treating a subject having chronic obstructive pulmonary disease (COPD) is also provided. The method comprises the step(s) of: delivering a therapeutically-effective amount of a pharmaceutical composition to the subject. The pharmaceutical composition comprises a cDNA fragment encoding BMI-1 and a pharmaceutically acceptable carrier which is pulmonary-targeted. The efficacy of the therapeutic method is demonstrated in the following experimental examples.

Because the composition, variation or connection relationship to other elements of each detail elements of the cDNA fragment encoding BMI-1 and the pharmaceutically acceptable carrier can refer to the previous embodiments, they are not repeated here.

Moreover, this embodiment also provides a use of a nucleic acid encoding BMI-1 for manufacturing a pharmaceutical composition for treating a subject having COPD. The composition, variation or connection relationship to other elements of each detail elements of the cDNA fragment encoding BMI-1 and the pharmaceutically acceptable carrier can refer to the previous embodiments, and they are not repeated here.

To illustrate the properties as well as the advantages of the embodiments described as above, there are several experimental examples shown below.

Animal Maintenance

Five-week-old Bltw:CD1 (ICR) mice are purchased from BioLasco Taiwan Co., Ltd. and maintained in the animal facility of Institute of Biomedical Sciences (IBMS), Academia Sinica. All animal experiment protocols are approved by the Institutional Animal Care and Use Committee of Academia Sinica (AS IACUC, Protocol ID: 11-03-161). Mice are maintained in controlled environmental conditions of temperature (22±2° C.) and humidity (60%±5%) with a strict 12 h light-dark cycle and with free access to food and water. Mice are subjected to experiments 1 week after receiving, with an average body weight around 20±2 g.

Plasmid Preparation

pPB-BMI1, with a sequence set forth as SEQ ID NO: 2, is a plasmid DNA containing human BMI1 cDNA sequence (set forth as SEQ ID NO: 1) under the transcriptional regulation of a CAG promoter. pPB-BMI1 is amplified in E. coli and purified using EndoFree Plasmid Mega Kit (Cat No./ID: 12381, Qiagen) according to the manufacturer's instructions.

PEI/DNA Nanoparticle Preparation

The PEI/DNA nanoparticle is prepared by in vivo jetPEI (#201, PolyPlus Transfection) with the manufacturer's instructions. Briefly, 30 μg of plasmid DNA is diluted in 5% glucose to a final volume of 100 μl in one tube. In another tube, 4.8 μl of in vivo jetPEI is diluted in 5% glucose to a final volume of 100 μl. The solution containing in vivo jetPEI is added to that containing DNA and mixed thoroughly by vortex for 10 s. The mixture is stood at room temperature for 20 minutes before injection.

Lung Emphysema and Therapy Model

To induce emphysema, mice are anesthetized by intraperitoneal injection with Zoletil (20 mg/kg) and Rompun (Xylazine 5 mg/kg) and intratracheally instilled with elastase (E7885, Sigma-Aldrich) at a dosage of 0.8 U/0.1 ml saline/mouse. After four or six hours later, mice, fully recovered from anesthesia, are intravenously injected with PEI/DNA nanoparticles through tail-vein. Later, mice are subjected to lung function analysis (non-invasive and/or invasive, at Week 4) and histopathological examination (at Week 4).

Lung Fibrosis and Therapy Model

To induce pulmonary fibrosis, mice are anesthetized by intraperitoneal injection with Zoletil (20 mg/kg) and Rompun (Xylazine 5 mg/kg) and intratracheally instilled with Bleomycin (1076308, Sigma-Aldrich) at a dosage of 0.06 mg/0.1 ml saline/mouse. Seven days later, mice are intravenously injected with PEI/DNA nanoparticles through tail-vein. Three weeks after Bleomycin administration, mice are subjected to lung function analysis (non-invasive and/or invasive) and histopathological examination.

Noninvasive Lung Function Analysis

Noninvasive lung function analysis is performed by using unrestrained whole-body plethysmography (WBP, Buxco system) following the manufacturer's instructions. In brief, mice are put in the whole-body plethysmograph cambers and allowed to move freely. With a constant flow of air through the chambers, the ventilatory parameters of mice, including respiratory rate (RR), tidal volume (TV), mid expiratory flow (EF50), inspiratory time (Ti), expiratory time (Te), peak inspiratory flow (PIF), peak expiratory flow (PEF), and enhanced pause (Penh) are measured and analyzed. The value of enhanced pause (Penh) is an indicator of bronchoconstriction and may represent the airway resistance of the mouse.

Invasive Lung Function Analysis

Mice are irreversibly anesthetized by intraperitoneal injection with Zoletil (200 mg/kg) and Rompun (Xylazine 50 mg/kg) and verified whether a surgical plane of anesthesia is reached by evaluating the toe pinch reflex. A lack of any observable response indicates that a surgical plane of anesthesia is reached. After the confirmation, the mouse's trachea is exposed for inserting an 18-gauge metal cannula and subjected to FlexiVent FX system (SCIREQ Inc., Montreal Qc, Canada) for invasive lung function analysis. The system is equipped with a FX1 module as well as NPFE extension for mice, operated by the flexiWare v7.2 software. Mice are quasi-sinusoidally ventilated with a tidal volume of 10 mL/kg, a frequency of 150 breaths/min, an inspiratory to expiratory ratio of 2:3, and a positive end-expiratory pressure of 3-cm H₂O. The measurement of lung function indexes, such as FEV0.1/FVC, FEV0.05/FVC, central airway resistance (Rn), tissue damping (G), tissue elastance (H), and tissue hypersensitivity (G/H), are performed according to manufacturer's instructions. FEV0.1 and FEV0.05 are the volume that has been exhaled at the end of the first 0.1 second and 0.05 second of forced expiration, respectively. FVC (forced vital capacity) is the vital capacity from a maximally forced expiratory effort.

Histopathological Analysis

After noninvasive or invasive lung function analysis, mice are perfused with 30 ml of PBS through heart. Lungs are removed, inflated and fixed by instilling 4% PFA from trachea, followed by immersion in 4% PFA at 4° C. before embedding in paraffin.

For investigation of emphysema severity, lungs are sectioned at a thickness of 5 μm and stained with Haematoxylin-eosin (HE) for histochemical examination. The area of alveolar space is estimated with Image-Pro Plus (Media Cybernetics, Inc.).

For investigation of fibrosis severity, lungs are sectioned at a thickness of 5μm and stained with Trichrome Staining (HT15, Sigma-Aldrich). The fibrotic (blue) regions are quantified by Image-Pro Plus.

### EXAMPLE 1: BMI-1 GENE DELIVERY BASED ON PEI NANOPARTICLES RESTORES PULMONARY FUNCTIONS IN MOUSE MODEL OF EMPHYSEMA

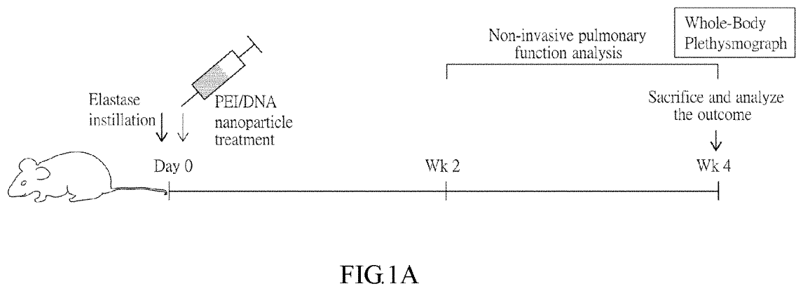

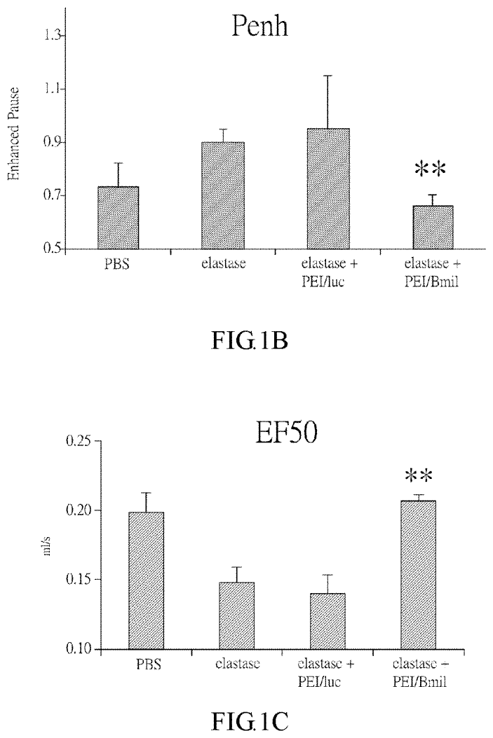

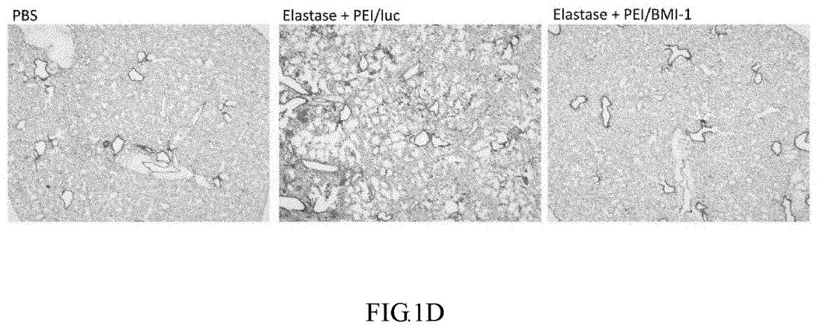

FIG. 1A represents a protocol, wherein mice are intratracheally instilled with elastase to induce the emphysema syndrome, and then treated with PEI/DNA nanoparticles for BMI-1 or control (luciferase, luc) gene delivery. Briefly, mice are divided into four groups: PBS (for blank control), elastase, elastase+PEI/luc, and elastase+PEI/BMI1. As described above, the mice are intratracheally instilled with elastase (for elastase, elastase+PEI/luc, and elastase+PEI/BMI1 groups) or PBS (for PBS group) at Day 0 to induce emphysema. After 4 hours, the mice in the elastase+PEI/luc and elastase+PEI/BMI1 groups are treated with PEI/DNA nanoparticles. At week 4, the mice of all groups are evaluated for pulmonary functions by using non-invasive whole-body plethysmographs, followed by histopathological analysis after being sacrificed.

Direct administration of elastase has been widely used to establish a mouse model of emphysema. With instillation of elastase (either intratracheal or intranasal), the enzyme will destruct lung tissues of the mice and cause the symptoms of abnormal pulmonary functions. As shown in FIGS. 1B and 1C, the intratracheally instillation of elastase actually induces impaired pulmonary functions in mice whereas intravenous injection of PEI/BMI-1 nanoparticles restores pulmonary functions of mice in the group of elastase+PEI/BMI1, as indicated by the value of Penh and EFS50. In addition, the histopathological results also show that the impairment of lung tissue induced by elastase is rescued by PEI/BMI-1 nanoparticles treatment.

### EXAMPLE 2: BMI-1 GENE DELIVERY BASED ON PEI NANOPARTICLES INDUCES SELF-RENEWAL OF ALVEOLAR EPITHELIAL CELLS (AECs) IN MOUSE MODEL OF EMPHYSEMA

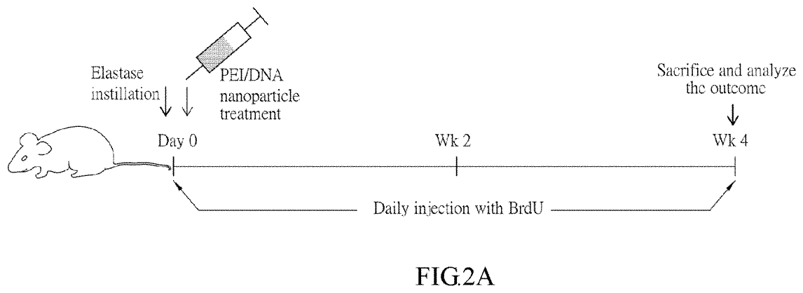

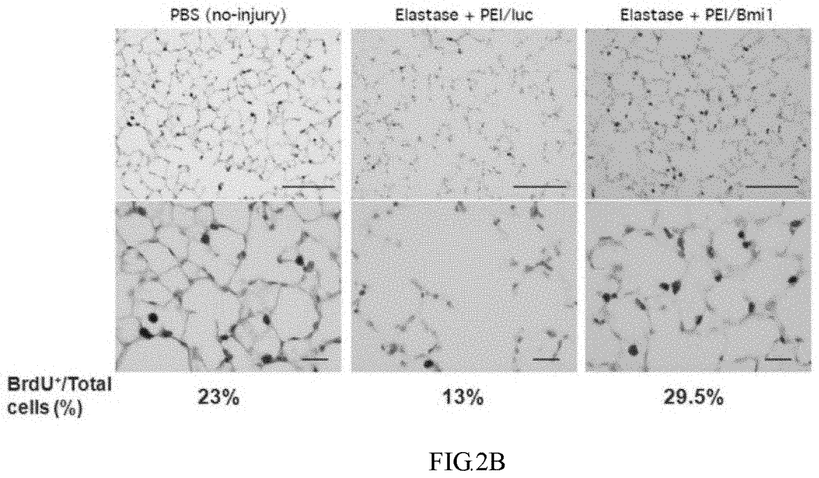

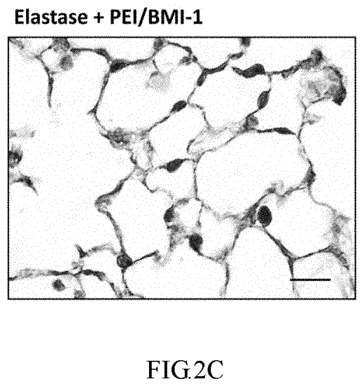

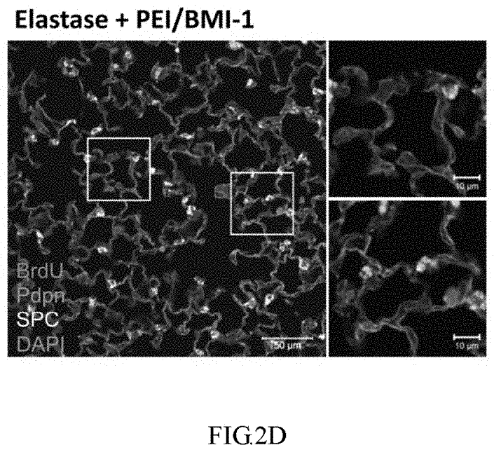

As shown in FIG. 2A, the mice are divided into three groups (PBS, elastase+PEI/luc, and elastase+PEI/MI1) and treated with the protocol substantially the same as the Example 1. In brief, the mice are also intratracheal instilled with elastase to establish the mouse model of COPD (i.e., to induce emphysema) and treated with PEI/DNA nanoparticles 4 hours post-injury. In addition, the mice are daily injected with 5-bromo-2′-deoxyuridine (BrdU) for a month. By Week 4, the mice are sacrificed and examined with IHC analysis for investigating proliferations of alveolar epithelial cells (AECs). The ratio of BrdU⁺/total cells are quantified from 30 photos (10 photos/mouse, 3 mice/group) for each group and indicated at the bottom of FIG. 2B.

As shown in FIG. 2C, the immunohistochemical (IHC) staining of BrdU confirms that mice treated with PEI/BMI-1 show much more proliferations of AECs compared to the control group PEI/luc (BrdU⁺/total cells: 29.5% vs. 13%). As shown in FIG. 2C, Verhoeff Van Gieson Elastin Staining shows that areas condensed with BrdU⁺ cells (the putative regenerated regions) have normal alveolar epithelial phenotype and ECM components without any symptoms of neoplasia. In FIG. 2D, the immunofluorescent (IF) staining is also performed to ensure that all BrdU⁺ cells showed AEC-1 or AEC-2 marker (Pdpn and SPC, respectively), suggesting the well differentiation of newly proliferated cells after treatment.

### EXAMPLE 3: PEI/BMI-1 NANOPARTICLES IMPROVE THE RATIO OF SLOW-CYCLING CELLS APPEARED POST-INJURY

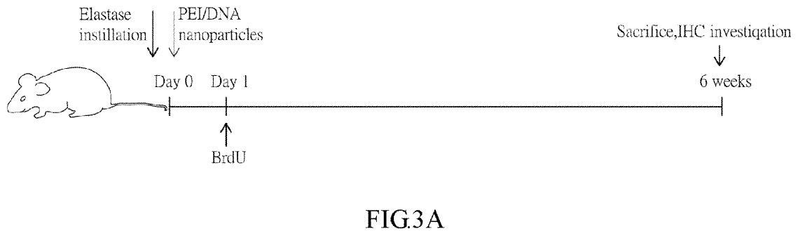

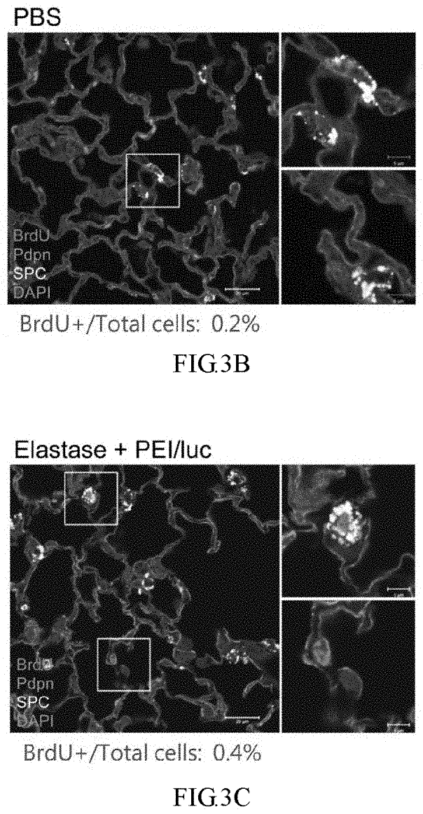

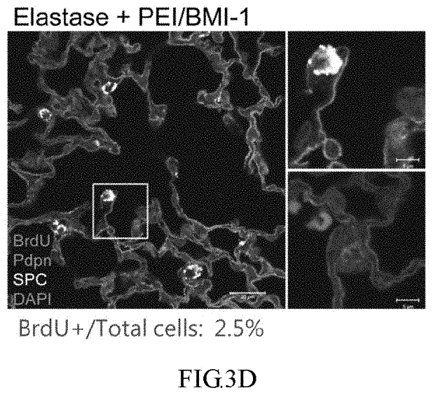

To evaluate whether PEI/BMI-1 mediates the reprogramming and re-acquisition of stemness property of target cells, the appearance of slow-cycling cells is investigated by injecting one single low-dose BrdU on day 1 post-injury and sacrificing the mice 6 weeks later for IHC staining with the protocol shown in FIG. 3A. As shown in FIGS. 3B to 3D, immunofluorescent (IF) staining of BrdU (red), Pdpn (green, AEC-1 marker), and SPC (white, AEC-2 marker) is performed in lung sections to verify the types and ratio of slow-cycling cells that appear after injury. The ratio of BrdU+/total cells are quantified from 30 photos (10 photos/mouse, 3 mice/group) for each group and indicated at the bottom of each figure. The results show that PEUBMI-1 indeed improves the ratio of slow-cycling cells labeled by BrdU in comparison with PEI/luc control group (BrdU⁺/total cells: 2.5% vs. 0.4%), suggesting that BMI-1 induces the self-renewal of target cells.

### EXAMPLE 4: DELIVERY OF PEUBMI-1 NANOPARTICLES SIGNIFICANTLY IMPROVES LUNG FUNCTIONS IN MICE WITH ELASTASE-INDUCED EMPHYSEMA

The mice are treated with the protocol substantially identical to that shown in FIG. 1A, wherein mice are intratracheally instilled with elastase to induced the emphysema syndrome and treated with PEI/DNA nanoparticles for BMI-1 or a control (GFP in the present embodiment) gene delivery. The mice used in the present experimental example are divided into three groups: NT (non-treatment, for blank control), PEI/GFP (non-therapy), and PEI/BMI1. The mice are intratracheally instilled with elastase (for PEI/GFP and PEI/BMI1 groups) or PBS (for NT group) at Day 0 to induce emphysema. 4 hours later, the mice in the groups of PEI/GFP and PEI/BMI1 are treated with PEI/DNA nanoparticles. At week 4, the mice are performed with invasive lung function analysis and sacrificed for histopathological analysis. For invasive lung function analysis, the ventilatory parameters of mice, including central airway resistance (Rn), tissue damping (G), tissue elastance (H), and tissue hypersensitivity (G/H), are measured and analyzed.

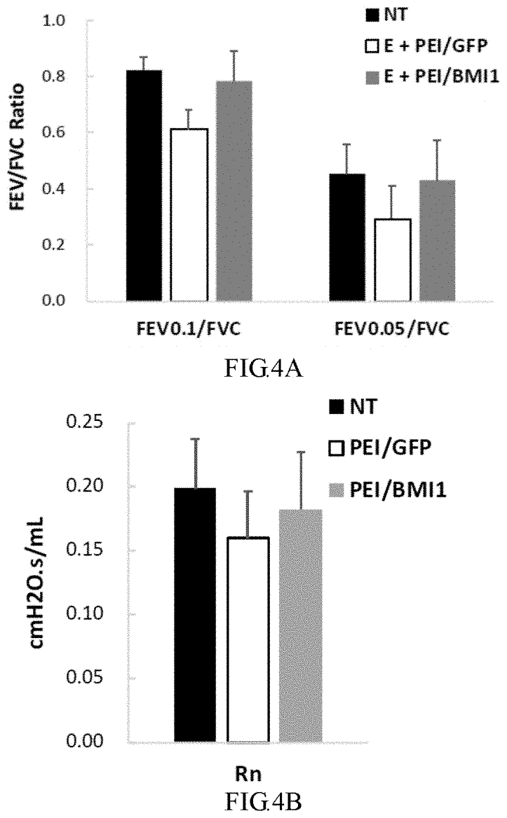

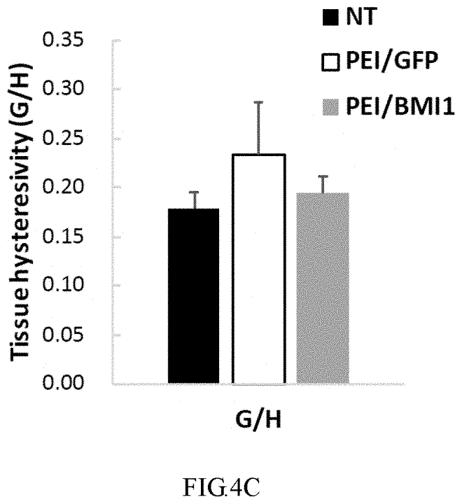

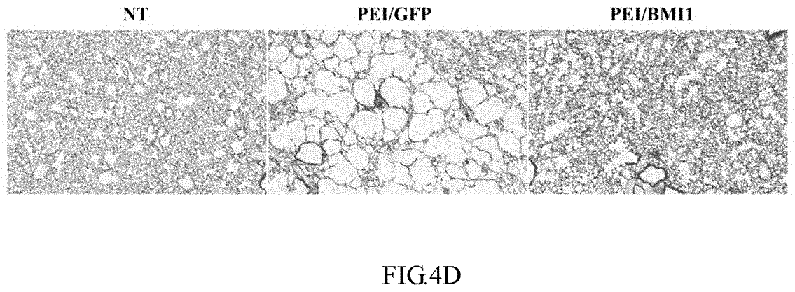

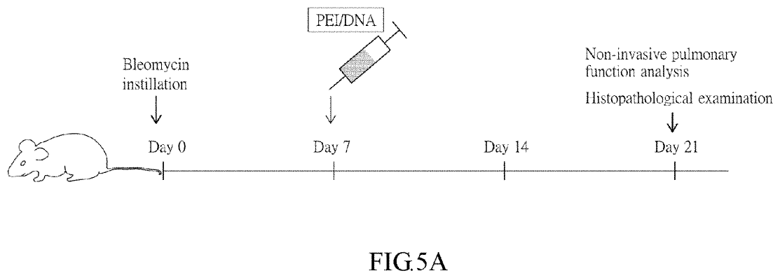

As shown in FIG. 4A, both the values of FEV0.1/FVC and FEV0.05/FVC, the clinic index of emphysema, are significantly reduced in the group treated with elastase and PEI/GFP (non-therapy group), suggesting the occurrence of emphysema. However, FEV0.1/FVC and FEV0.05/FVC value of mice treated with elastase and PEI/BMI1 are restored to the level close to NT group, suggesting a significant recovery from emphysema syndrome. From FIGS. 4B and 4C, the central airway resistance (Rn, shown in FIG. 4B) and the tissue hypersensitivity (G/H, shown in FIG. 4C) are both improved by BMI-1 gene delivery. Collectively, delivery of BMI-1 gene using PEUBMI-1 nanoparticles actually restores pulmonary functions of mice. In addition, the histopathological results also validates that the impairment of lung tissue induced by elastase is rescued by PEUBMI-1 nanoparticles treatment.

### EXAMPLE 5: DELIVERY OF PEUBMI-1 NANOPARTICLES SIGNIFICANTLY IMPROVES LUNG FUNCTIONS IN MICE WITH PULMONARY FIBROSIS INDUCED BY BLEOMYCIN

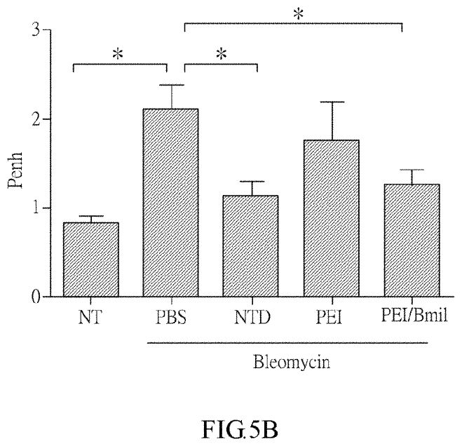

As shown in FIG. 5A, the mice are divided into five groups, and at Day 0 the mice are treated with a protocol of intratracheal Bleomycin instillation to induce lung fibrosis or with PBS (for NT group), a positive blank control. At Day 7, the mice treated with Bleomycin are intravenously injected with PBS (for PBS group, as a non-therapy control), nintedanib (for NTD group, as a positive therapy control), PEI nanoparticles only (for PEI group, as a mock-therapy control), and PEI/BMI-1 nanoparticles (for PEI/BMI1 group). Two weeks after PEI nanoparticles or nintedanib treatment (i.e., at Day 21), the mice are all performed with noninvasive lung function analysis using unstrained whole-body plethysmography as described above, followed by histological examination after sacrifice. Nintedanib (NTD) is known for its anti-fibrotic effects in patients with idiopathic pulmonary fibrosis and used as a positive control for pulmonary fibrosis treatment.

Bleomycin has been used to develop animal model of pulmonary fibrosis as a useful tool to investigate the efficacy of a potential treatment. The mice with Bleomycin treatment will have pulmonary fibrosis and symptoms of impaired pulmonary functions. As shown in FIG. 5B, the airway resistance of the mice in PBS group (only with IT instillation of Bleomycin but without PEI nanoparticle treatment), are found to be significantly higher (*p<0.05) than those in NT group, suggesting Bleomycin actually impairs the lung functions of the mice. In the meantime, the airway resistances (as indicated by Penh value) of the mice in PEI/BMI1 group are found to be significantly reduced (*p<0.05, compared with PBS group) and close to the levels of NT (non-treatment) and NTD (positive therapy control) groups, whereas the mice treated with PEI nanoparticle (mock-therapy) are found to have little change in airway resistances which are not statistically different from those in PBS group. The results suggest that the PEI/BMI-1 treatment restors the impaired lung functions caused by Bleomycin, and its efficacy is as efficiently as NTD treatment

In summary, the present disclosure provides a nucleic acid fragment, a pharmaceutical composition, and a therapeutic process for treating a subject having chronic obstructive pulmonary disease (COPD). Especially, the nucleic acid fragment, the pharmaceutical composition, and the therapeutic process are therapeutically efficient for treating pulmonary fibrosis and emphysema of the subject, as demonstrated in this disclosure. In the aforementioned experiments, BMI-1 gene delivery by using PEI/DNA nanoparticles can restore the impaired pulmonary functions caused by elastase-induced emphysema and Bleomycin-induced pulmonary fibrosis proved by the results of either noninvasive or invasive lung function analysis or the histological examinations. Also, it is found that BMI-1 cDNA fragment induces the proliferation and differentiation of alveolar epithelial cells (AECs) by improving the ratio of slow-cycling cells appeared post-injury.

Although the invention has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the invention.

What is claimed is:

1. A nucleic acid fragment for treating a subject having chronic obstructive pulmonary disease (COPD), comprising a cDNA fragment encoding BMI-1.

2. The nucleic acid fragment according to claim 1, wherein the cDNA fragment encodes human BMI-1.

3. The nucleic acid fragment according to claim 2, wherein the cDNA fragment has a sequence of SEQ ID NO: 1.

4. The nucleic acid fragment according to claim 3, wherein the cDNA fragment has a sequence of SEQ ID NO: 2.

5. The nucleic acid fragment according to claim 1, wherein the subject is a mammal.

6. The nucleic acid fragment according to claim 1, wherein the subject is human, primate, hamster, rabbit, rodent, bovine, swine, sheep, horse, goat, canine or feline.

7. The nucleic acid fragment according to claim 1, wherein the nucleic acid fragment relieves or ameliorates emphysema of the subject.

8. The nucleic acid fragment according to claim 1, wherein the nucleic acid fragment relieves or ameliorates pulmonary fibrosis of the subject.

9. A pharmaceutical composition for treating a subject having chronic obstructive pulmonary disease (COPD), comprising:
a cDNA fragment encoding BMI-1; and
a pharmaceutically acceptable carrier which is pulmonary-targeted.

10. The pharmaceutical composition according to claim 9, wherein the pharmaceutically acceptable carrier is a cationic polymer with a molar formula of (C₂H₅N)ₙ, wherein n is an integer of about 10 to 1000.

11. The pharmaceutical composition according to claim 10, wherein the pharmaceutically acceptable carrier is polyethylenimine.

12. The pharmaceutical composition according to claim 9, wherein the cDNA fragment encodes human BMI-1.

13. The pharmaceutical composition according to claim 12, wherein the cDNA fragment has a sequence of SEQ ID NO: 1.

14. The pharmaceutical composition according to claim 13, wherein the cDNA fragment has a sequence of SEQ ID NO: 2.

15. The pharmaceutical composition according to claim 9, wherein the subject is a mammal.

16. The pharmaceutical composition according to claim 9, wherein the subject is human, primate, hamster, rabbit, rodent, bovine, swine, sheep, horse, goat, canine or feline.

17. The pharmaceutical composition according to claim 9, wherein the pharmaceutical composition relieves or ameliorates emphysema of the subject.

18. The pharmaceutical composition according to claim 9, wherein the pharmaceutical composition relieves or ameliorates pulmonary fibrosis of the subject.

19. A method for treating a subject having chronic obstructive pulmonary disease (COPD), comprising:
delivering a therapeutically effective amount of a pharmaceutical composition to the subject, which comprises:
a cDNA fragment encoding BMI-1; and
a pharmaceutically acceptable carrier which is pulmonary-targeted.

20. The method according to claim 19, wherein the pharmaceutically acceptable carrier is a cationic polymer with a molar formula of (C₂H₅N)ₙ, wherein n is an integer of about 10 to 1000.

21. The method according to claim 19, wherein the pharmaceutically acceptable carrier is polyethylenimine.

22. The method according to claim 19, wherein the cDNA fragment encodes human BMI-1.

23. The method according to claim 22, wherein the cDNA fragment has a sequence of SEQ ID NO: 1.

24. The method according to claim 23, wherein the cDNA fragment has a sequence of SEQ ID NO: 2.

25. The method according to claim 19, wherein the subject is a mammal.

26. The method according to claim 19, wherein the subject is human, primate, hamster, rabbit, rodent, bovine, swine, sheep, horse, goat, canine or feline.

27. The method according to claim 19, wherein the pharmaceutical composition relieves or ameliorates emphysema of the subject.

28. The method according to claim 19, wherein the pharmaceutical composition relieves or ameliorates pulmonary fibrosis of the subject.
